(19) United States
(12) Patent Application Publication
(10) Pub. No.: US 2017/0234783 A1
(43) Pub. Date: Aug. 17, 2017

(54) BIOLOGICAL SUBSTANCE EXTRACTION DEVICE AND BIOLOGICAL SUBSTANCE EXTRACTION APPARATUS

(71) Applicant: SEIKO EPSON CORPORATION, Tokyo (JP)

(72) Inventor: Toshiro MURAYAMA, Fujimi-machi (JP)

(21) Appl. No.: 15/514,700

(22) PCT Filed: Sep. 30, 2015
(86) PCT No.: PCT/JP2015/004978

§ 371 Date: Mar. 27, 2017

(30) Foreign Application Priority Data
Sep. 30, 2014 (JP) 2014-199565

(51) Int. Cl. G01N 1/40; B01L 3/00; C12N 15/10; C12Q 1/68

(52) U.S. Cl. CPC G01N 1/405; C12Q 1/6806; B01L 3/502; C12N 15/1013; B01L 2300/0867; B01L 2300/0848; B01L 2200/0631; B01L 2300/0609; G01N 2001/4038; B01L 2200/0689

(57) ABSTRACT

A biological substance extraction device includes an adsorption container that includes a first flow channel, and seal-tightly holds an adsorbent and a fluid within the first flow channel, and a washing container that includes a second flow channel, and seal-tightly holds a washing liquid and a fluid within the second flow channel, the adsorption container and the washing container being joined to form a flow channel through which a biological substance is moved. The first flow channel and the second flow channel communicate with each other in a state in which an insertion section is inserted into a reception section. The insertion section includes a guide member that extends from the first flow channel to the second flow channel. The guide member forms part of the flow channel between a first inner wall of the first flow channel and a second inner wall of the second flow channel.

### CROSS REFERENCE TO RELATED APPLICATIONS

This application is a U.S. National Phase Application under 35 U.S.C. 371 of International Application No. PCT/JP2015/004978 filed on Sep. 30, 2015 and published in English as WO 2016/051795 A1 on Apr. 7, 2016 and claims priority to Japanese Patent Application No. 2014-199565 filed on Sep. 30, 2014. The entire disclosures of all of the above applications are incorporated herein by reference.

### TECHNICAL FIELD

The present invention relates to a biological substance extraction device and a biological substance extraction apparatus.

### BACKGROUND ART

Polymerase chain reaction (PCR) technology has been established in the field of biochemistry. In recent years, PCR amplification accuracy and PCR detection sensitivity have been improved, and it has become possible to amplify, detect, and analyze a trace amount of a sample (e.g., DNA). PCR technology subjects a solution (reaction solution) that includes the amplification target nucleic acid (target nucleic acid) and a reagent to thermal cycling to amplify the target nucleic acid. The solution is normally subjected to PCR thermal cycling at two or three different temperatures.

At present, the presence or absence of infection (e.g., influenza) is normally determined using a rapid test kit (e.g., immunochromatography). However, since the determination accuracy may be insufficient when such a rapid test kit is used, it has been desired to use PCR technology that can achieve higher examination accuracy when determining the presence or absence of infection.

In recent years, a device in which aqueous liquid layers and water-insoluble gel layers are alternately stacked within a capillary has been proposed as a device used for PCR technology and the like (see WO2012/086243). In this case, a magnetic material particle to which a nucleic acid adheres is passed through the capillary to purify the nucleic acid. However, such a device has a problem in that a component of one aqueous liquid layer may gradually diffuse through the gel layer, and contaminate another aqueous liquid layer when stored for a long time.

### SUMMARY OF INVENTION

### Technical Problem

An object of the present invention is to provide a biological substance extraction device and a biological substance extraction apparatus that make it possible to move a substance-binding solid-phase carrier by applying a magnetic force even when a step is formed on the inner wall of a flow channel.

### Solution to Problem

The invention was conceived in order to solve at least some of the above problems, and may be implemented as described below (see the following aspects and application examples).

### Application Example 1

According to one aspect of the invention, a biological substance extraction device includes a flow channel through which a biological substance is moved, the flow channel being formed by joining a first container that includes a first flow channel and seal-tightly holds a first liquid and a fluid that is immiscible with the first liquid within the first flow channel, and a second container that includes a second flow channel and seal-tightly holds a second liquid and a fluid that is immiscible with the second liquid within the second flow channel,

one end of the first flow channel being inserted into one end of the second flow channel so that the first flow channel and the second flow channel communicate with each other,
the first container including a guide member that extends from the first flow channel to the second flow channel when the first flow channel and the second flow channel communicate with each other, and
the guide member forming part of the flow channel between a first inner wall of the first flow channel and a second inner wall of the second flow channel.

According to the biological substance extraction device, it is possible to move the substance-binding solid-phase carrier from the second flow channel within the second container to the first flow channel within the first container even in a state in which one end of the first flow channel is inserted into one end of the second flow channel by guiding the substance-binding solid-phase carrier using the guide member.

### Application Example 2

In the biological substance extraction device, the guide member may have a plate-like shape, and a plurality of the guide members may be provided to intersect each other.

According to this configuration, it is possible to improve the degree of freedom relating to the phase (phase control) in the circumferential direction around the flow channel of a washing container with respect to an adsorption container when joining the adsorption container and the washing container.

### Application Example 3

In the biological substance extraction device, a substance-binding solid-phase carrier may be provided on the downstream side of the guide member within the flow channel through which the biological substance is moved.

According to this configuration, it is possible to guide the substance-binding solid-phase carrier provided on the downstream side of the guide member to the first flow channel using the guide member.

### Application Example 4

In the biological substance extraction device, the first container may be an adsorption container, the second container may be a washing container, the first liquid may be an adsorbent, and the second liquid may be a washing liquid. According to this configuration, it is possible to guide the substance-binding solid-phase carrier from the washing container to the adsorption container using the guide member after the flow has been formed.

### Application Example 5

According to another aspect of the invention, a biological substance extraction apparatus includes:

a holding section that holds the biological substance extraction device; and
a magnet moving mechanism that moves a magnet along the biological substance extraction device that is held by the holding section,
the magnet moving mechanism moving a substance-binding solid-phase carrier provided within the washing container to the adsorption container along the guide member by moving the magnet.

According to the biological substance extraction apparatus, it is possible to move the substance-binding solid-phase carrier from the second flow channel to the first flow channel by causing the magnet moving mechanism to move the substance-binding solid-phase carrier along the guide member by moving the magnet.

### Application Example 6

In the biological substance extraction apparatus, the biological substance extraction device may further include an elution container that is connected to the other end of the second flow channel, the elution container may hold an eluent that is a liquid with which the biological substance is eluted from the substance-binding solid-phase carrier, and the magnet moving mechanism may move the substance-binding solid-phase carrier through the adsorption container, the washing container, and the elution container along the flow channel by moving the magnet to elute the biological substance from the substance-binding solid-phase carrier.

According to this configuration, the biological substance can be eluted from the substance-binding solid-phase carrier by causing the biological substance to be adsorbed on the substance-binding solid-phase carrier that has moved to the adsorption container along the guide member, and then moving the substance-binding solid-phase carrier along the flow channel within the washing container and the elution container using the magnet moving mechanism.

### BRIEF DESCRIPTION OF DRAWINGS

FIG. 1 is a front view illustrating a container assembly 1 according to one embodiment of the invention.

FIG. 2 is a side view illustrating a container assembly 1 according to one embodiment of the invention.

FIG. 3 is a plan view illustrating a container assembly 1 according to one embodiment of the invention.

FIG. 4 is a perspective view illustrating a container assembly 1 according to one embodiment of the invention.

FIG. 5 is a cross-sectional view illustrating a container assembly 1 according to one embodiment of the invention taken along the line A-A illustrated in FIG. 3.

FIG. 6 is a cross-sectional view illustrating a container assembly 1 according to one embodiment of the invention taken along the line C-C illustrated in FIG. 3.

FIG. 7A is a schematic view illustrating a method for operating a container assembly 1 according to one embodiment of the invention.

FIG. 7B is a schematic view illustrating a method for operating a container assembly 1 according to one embodiment of the invention.

FIG. 8A is a schematic view illustrating a method for operating a container assembly 1 according to one embodiment of the invention.

FIG. 8B is a schematic view illustrating a method for operating a container assembly 1 according to one embodiment of the invention.

FIG. 9 is a schematic configuration diagram illustrating a PCR device 50.

FIG. 10 is a block diagram illustrating a PCR device 50.

FIG. 11 is a plan view illustrating a nucleic acid extraction device 6 according to one embodiment of the invention.

FIG. 12 is a cross-sectional view illustrating a nucleic acid extraction device 6 according to one embodiment of the invention taken along the line C-C illustrated in FIG. 11.

FIG. 13 is a vertical cross-sectional view illustrating an adsorption container 100 taken along the line C-C illustrated in FIG. 11.

FIG. 14 is a vertical cross-sectional view illustrating a first washing container 210 taken along the line C-C illustrated in FIG. 11.

FIG. 15 is a perspective view illustrating a first washing container 210.

FIG. 16 is a vertical cross-sectional view illustrating a second washing container 220 taken along the line C-C illustrated in FIG. 11.

FIG. 17 is a schematic view illustrating a method for operating a nucleic acid extraction device 6 according to one embodiment of the invention.

FIG. 18 is a schematic view illustrating a method for operating a nucleic acid extraction device 6 according to one embodiment of the invention.

FIG. 19 is a schematic view illustrating a method for operating a nucleic acid extraction device 6 according to one embodiment of the invention.

FIG. 20 is a schematic view illustrating a method for operating a nucleic acid extraction device 6 according to one embodiment of the invention.

FIG. 21 is a block diagram illustrating a nucleic acid extraction apparatus 50A according to one embodiment of the invention.

FIG. 22 is a side view illustrating a nucleic acid extraction apparatus 50A according to one embodiment of the invention.

### DESCRIPTION OF EMBODIMENTS

Several exemplary embodiments of the invention are described below. Note that the following exemplary embodiments merely illustrate examples of the invention. The invention is not limited to the following exemplary embodiments. The invention includes various modifications that can be practiced without departing from the scope of the invention. Note that all of the elements described below in connection with the exemplary embodiments should not necessarily be taken as essential elements of the invention.

According to one embodiment of the invention, a biological substance extraction device includes a flow channel through which a biological substance is moved, the flow channel being formed by joining a first container that includes a first flow channel and seal-tightly holds a first liquid and a fluid that is immiscible with the first liquid within the first flow channel, and a second container that includes a second flow channel and seal-tightly holds a second liquid and a fluid that is immiscible with the second liquid within the second flow channel, one end of the first flow channel being inserted into one end of the second flow channel so that the first flow channel and the second flow channel communicate with each other, the first container including a guide member that extends from the first flow channel to the second flow channel when the first flow channel and the second flow channel communicate with each other, and the guide member forming part of the flow channel between a first inner wall of the first flow channel and a second inner wall of the second flow channel.

A biological substance extraction apparatus according to one embodiment of the invention includes a holding section that holds the biological substance extraction device, and a magnet moving mechanism that moves a magnet along the biological substance extraction device that is held by the holding section, the magnet moving mechanism moving a substance-binding solid-phase carrier provided within the washing container to the adsorption container along the guide member by moving the magnet.

An embodiment in which a container assembly 1 is used as the biological substance extraction device will be described first, and an embodiment in which a PCR device 50 is used as the biological substance extraction apparatus will then be described. The details of the guide member are described later in section “5. Nucleic acid extraction device”.

Examples of the biological substance include a biopolymer such as a nucleic acid (DNA and RNA), a polypeptide, a protein, and a polysaccharide, a biological low-molecular-weight organic compound such as a protein, an enzyme, a peptide, a nucleotide, an amino acid, and a vitamin, an inorganic compound, and the like. The embodiments of the invention will be described taking an example in which the biological substance is a nucleic acid.

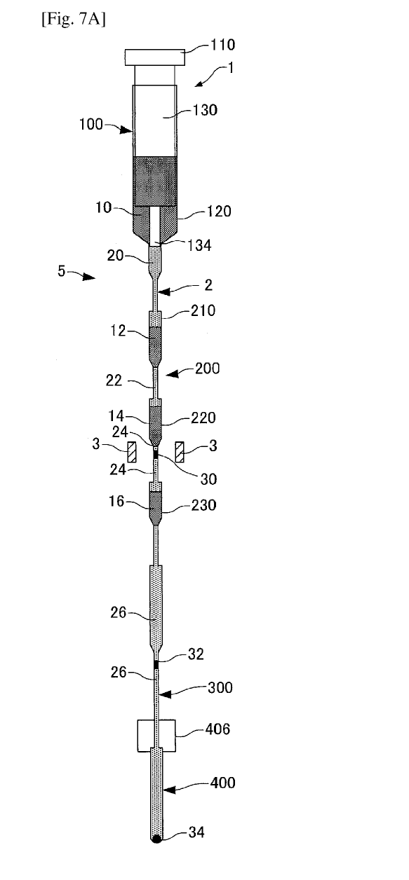

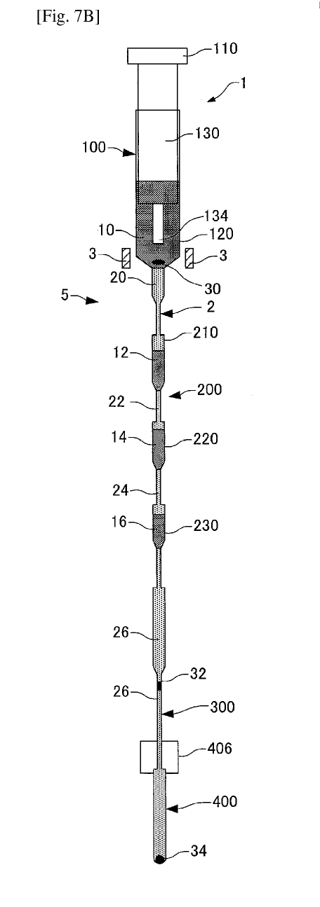

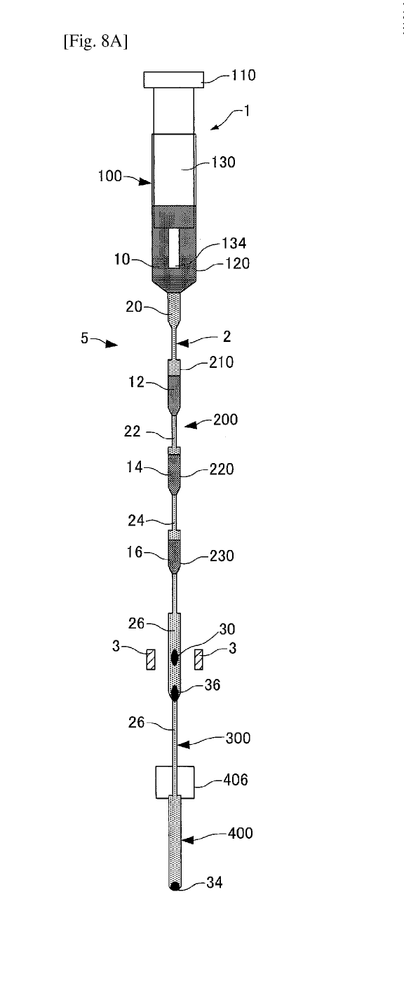

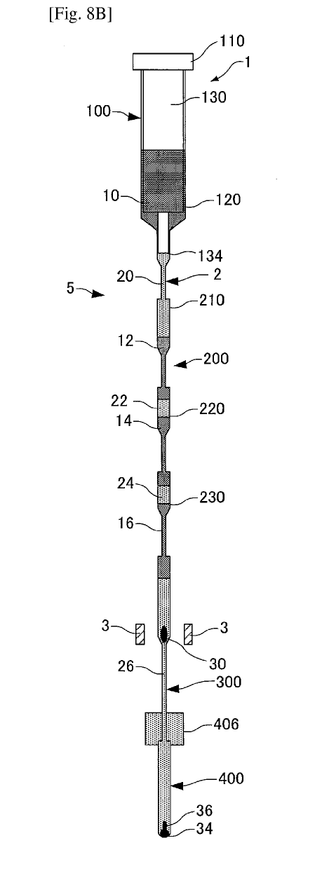

The term “substance-binding solid-phase carrier” used herein refers to a substance that can hold the biological substance through adsorption (i.e., reversible physical binding). It is preferable that the substance-binding solid-phase carrier be microparticles. Note that the substance-binding solid-phase carrier is not limited thereto. For example, the substance-binding solid-phase carrier may be microfibers or a net-like carrier. It is preferable that the substance-binding solid-phase carrier have magnetic properties so that the substance-binding solid-phase carrier can be moved in the desired direction within the container assembly in a state in which the biological substance is adsorbed on the substance-binding solid-phase carrier. The embodiments of the invention will be described taking an example in which the substance-binding solid-phase carrier is a magnetic bead 30 (see FIGS. 7A, 7B, 8A, and 8B) on which a nucleic acid is adsorbed.

The washing liquid 12, 14, 16 (see FIGS. 7A, 7B, 8A, and 8B) is a liquid for washing the substance-binding solid-phase carrier on which the biological substance is adsorbed. It is possible to remove impurities and the like while ensuring that the biological substance is adsorbed on the substance-binding solid-phase carrier in a stable manner by washing the substance-binding solid-phase carrier with the washing liquid.

The fluid that is immiscible with the washing liquid is a fluid that is immiscible with the washing liquid within the washing container, and undergoes phase separation with respect to the washing liquid. The fluid that is immiscible with the washing liquid is a substance that is inert to the washing liquid, and may be a gas such as air. When the washing liquid is an aqueous liquid, an oil, an oil gel, or the like that is immiscible with the aqueous liquid may be used as the fluid that is immiscible with the washing liquid. The term “oil gel” used herein refers to a gel that is obtained by subjecting a liquid oil to gelation using a gellant. Note that the term “oil” used herein excludes an oil gel. The embodiments of the invention will be described taking an example in which the fluid that is immiscible with the washing liquid is an oil 20, 22, 24, 26 (see FIGS. 7A, 7B, 8A, and 8B).

The eluent 32 (see FIGS. 7A, 7B, 8A, and 8B) is a substance with which the biological substance is desorbed and eluted from the substance-binding solid-phase carrier. For example, water or a buffer may be used as the eluent.

The fluid that is immiscible with the eluent is a fluid that is immiscible with the eluent within the elution container, and undergoes phase separation with respect to the eluent. The fluid that is immiscible with the eluent is a substance that is inert to the eluent. The embodiments of the invention will be described taking an example in which the fluid that is immiscible with the eluent is an oil 26 (see FIGS. 7A, 7B, 8A, and 8B).

### 1. Outline of Container Assembly

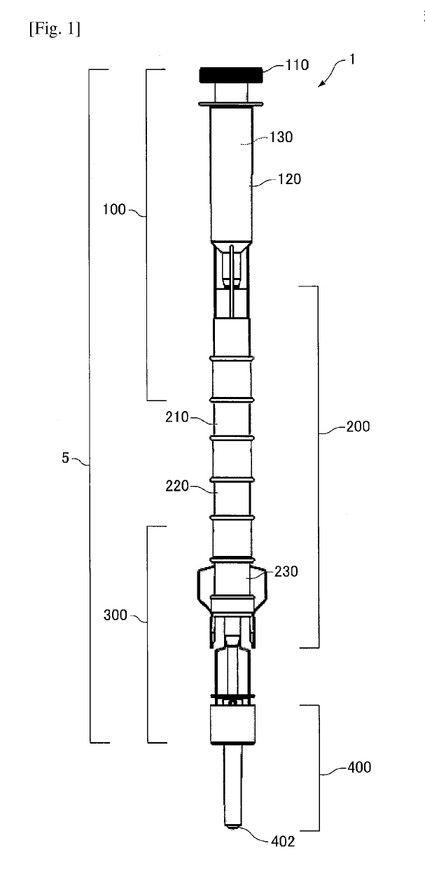

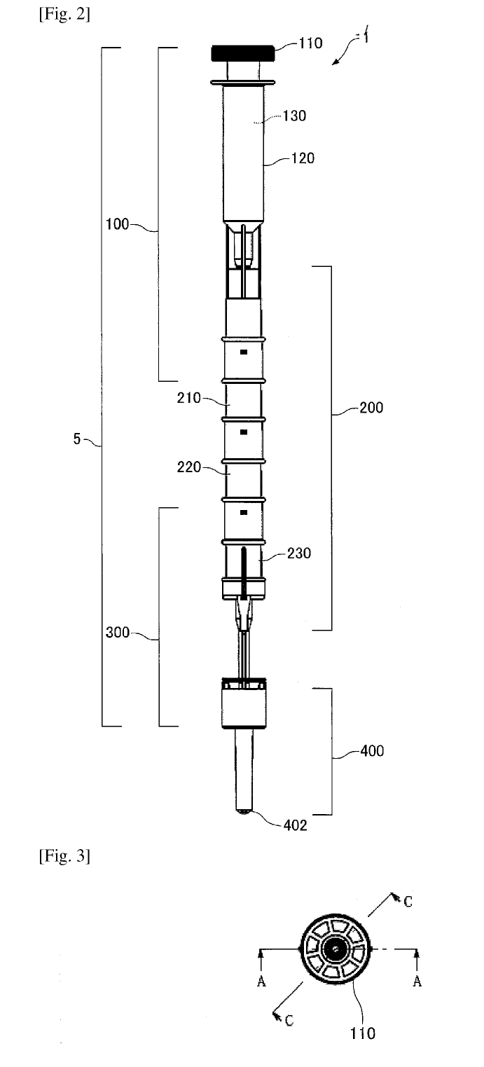

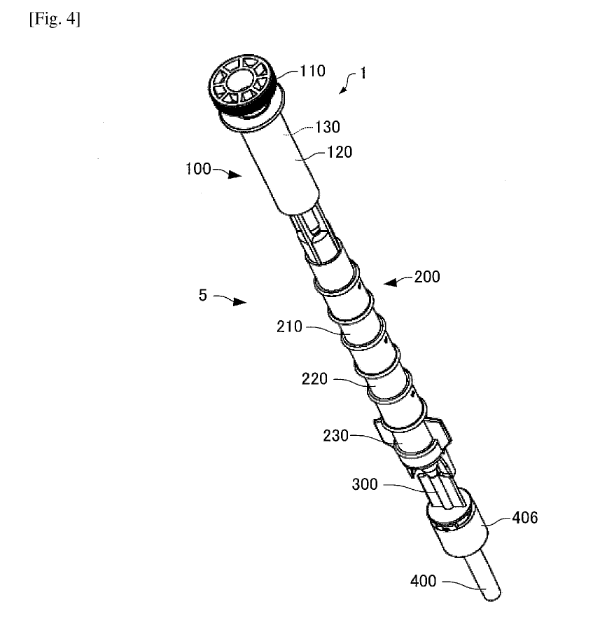

An outline of a container assembly 1 according to one embodiment of the invention is described below with reference to FIGS. 1 to 4. FIG. 1 is a front view illustrating the container assembly 1 (hereinafter may be referred to as “cartridge”) according to one embodiment of the invention. FIG. 2 is a side view illustrating the container assembly 1 according to one embodiment of the invention. FIG. 3 is a plan view illustrating the container assembly 1 according to one embodiment of the invention. FIG. 4 is a perspective view illustrating the container assembly 1 according to one embodiment of the invention. Note that the state of the container assembly 1 illustrated in FIGS. 1 to 3 is referred to as “upright state”.

The container assembly 1 includes an adsorption container 100, a washing container 200, an elution container 300, and a reaction container 400. The container assembly 1 is a container that forms a flow channel (not illustrated in the drawings) that extends (communicates) from the adsorption container 100 to the reaction container 400. The flow channel formed by the container assembly 1 is closed by a cap 110 at one end, and is closed by a bottom 402 at the other end.

The container assembly 1 is designed to effect a pretreatment that causes a nucleic acid to be bound to a magnetic bead (not illustrated in the drawings) within the adsorption container 100, purified while the magnetic bead moves within the washing container 200, and eluted into an eluent droplet (not illustrated in the drawings) within the elution container 300, and subjects the eluent droplet that includes the nucleic acid to PCR thermal cycling within the reaction container 400.

A material for forming the container assembly 1 is not particularly limited. For example, the container assembly 1 may be formed of glass, a polymer, a metal, or the like. It is preferable to form the container assembly 1 using a material (e.g., glass or polymer) that allows visible light to pass through since the inside (cavity) of the container assembly 1 can be observed from the outside. It is preferable to form the container assembly 1 using a material that allows a magnetic force to pass through or a non-magnetic material since the magnetic bead (not illustrated in the drawings) can be easily passed through the container assembly 1 by applying a magnetic force from the outside of the container assembly 1, for example. The container assembly 1 may be formed of a polypropylene resin, for example.

The adsorption container 100 includes a cylindrical syringe section 120 that holds an adsorbent (not illustrated in the drawings), a plunger section 130 that is a movable plunger that is inserted into the syringe section 120, and the cap 110 that is secured on one end of the plunger section 130. The adsorption container 100 is designed so that the plunger section 130 can be slid along the inner surface of the syringe section 120, and the adsorbent (not illustrated in the drawings) contained in the syringe section 120 can be discharged into the washing container 200 by moving the cap 110 toward the syringe section 120. The details of the adsorbent are described later.

The washing container 200 is assembled by joining a first washing container 210, a second washing container 220, and a third washing container 230. Each of the first washing container 210, the second washing container 220, and the third washing container 230 includes one or more washing liquid layers that are partitioned by an oil layer (not illustrated in the drawings). The washing container 200 (assembled by joining the first washing container 210, the second washing container 220, and the third washing container 230) includes a plurality of washing liquid layers that are partitioned by a plurality of oil layers (not illustrated in the drawings). Although an example in which the washing container 200 utilizes the first washing container 210, the second washing container 220, and the third washing container 230 has been described above, the number of washing containers may be appropriately increased or decreased corresponding to the number of washing liquid layers. The details of the washing liquid are described later.

The elution container 300 is joined to the third washing container 230 included in the washing container 200, and holds the eluent so that the shape of a plug can be maintained. The term “plug” used herein refers to a specific liquid when the specific liquid occupies a space (compartment) within a flow channel. More specifically, the plug of a specific liquid refers to a pillar-shaped space that is substantially occupied by only the specific liquid (i.e., the space within the flow channel is partitioned by the plug of the liquid). The expression “substantially” used in connection with the plug means that a small amount (e.g., thin film) of another substance (e.g., liquid) may be present around the plug (i.e., on the inner wall of the flow channel). The details of the eluent are described later.

A nucleic acid purification device 5 includes the adsorption container 100, the washing container 200, and the elution container 300.

The reaction container 400 is joined to the elution container 300, and receives a liquid discharged from the elution container 300. The reaction container 400 holds the eluent droplet that includes a sample during thermal cycling. The reaction container 400 also holds a reagent (not illustrated in the drawings). The details of the reagent are described later.

### 2. Details of Structure of Container Assembly

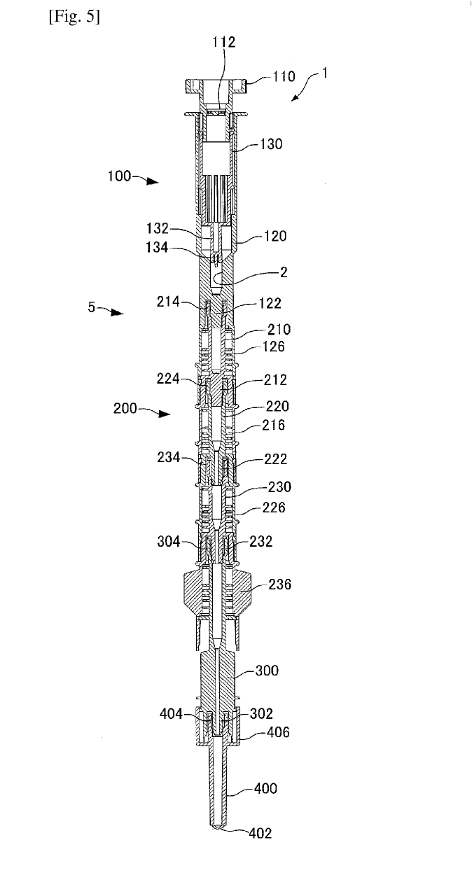

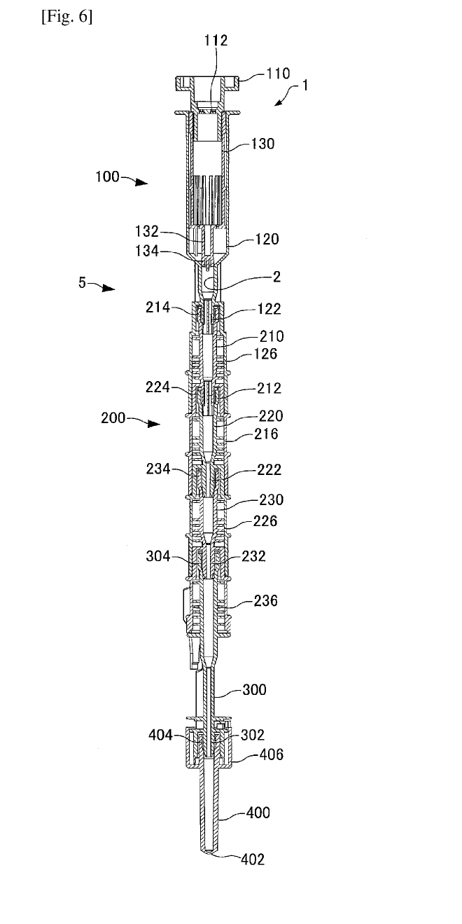

The details of the structure of the container assembly 1 are described below with reference to FIGS. 5 and 6. FIG. 5 is a cross-sectional view of the container assembly 1 according to one embodiment of the invention taken along the line A-A in FIG. 3. FIG. 6 is a cross-sectional view of the container assembly 1 according to one embodiment of the invention taken along the line C-C in FIG. 3. Note that the container assembly 1 is assembled in a state in which each container is charged with the washing liquid or the like. In FIGS. 5 and 6, the washing liquid and the like are omitted so that the structure of the container assembly 1 can be easily understood.

### 2-1. Adsorption Container

The adsorption container 100 has a structure in which the plunger section 130 is inserted into the syringe section 120 through one open end of the syringe section 120, and the cap 110 is inserted into the open end of the plunger section 130. The cap 110 has a vent section 112 that is provided at the center thereof. The vent section 112 suppresses a change in the internal pressure of the plunger section 130 when the plunger section 130 is operated.

The plunger section 130 is an approximately cylindrical plunger that slides along the inner circumferential surface of the syringe section 120. The plunger section 130 includes the open end into which the cap 110 is inserted, a rod-like section 132 that extends from the bottom situated opposite to the open end in the longitudinal direction of the syringe section 120, and an end section 134 that is provided at the end of the rod-like section 132. The rod-like section 132 protrudes from the center of the bottom of the plunger section 130. A through-hole is formed in the wall of the rod-like section 132 so that the inner space of the plunger section 130 communicates with the inner space of the syringe section 120.

The syringe section 120 forms part of a flow channel 2 of the container assembly 1. The syringe section 120 includes a large-diameter section that holds the plunger section 130, a small-diameter section that is smaller in inner diameter than the large-diameter section, a diameter reduction section that is provided between the large-diameter section and the small-diameter section and decreases in inner diameter, an adsorption insertion section 122 that is provided at the end of the small-diameter section, and a cylindrical adsorption cover section 126 that covers the adsorption insertion section 122. The large-diameter section, the small-diameter section, and the adsorption insertion section 122 that form part of the flow channel 2 of the container assembly 1 have an approximately cylindrical shape.

The end section 134 of the plunger section 130 seals the small-diameter section of the syringe section 120 (when the container assembly 1 is provided to the worker) to divide the large-diameter section and the diameter reduction section from the small-diameter section (i.e., divide the syringe section 120 into two compartments).

The adsorption insertion section 122 of the syringe section 120 is inserted and fitted into a first reception section 214 that forms one open end of the first washing container 210 included in the washing container 200 to join the syringe section 120 and the first washing container 210. The outer circumferential surface of the adsorption insertion section 122 comes in close contact with the inner circumferential surface of the first reception section 214 to prevent leakage of a liquid to the outside.

### 2-2. Washing Container

The washing container 200 forms part of the flow channel 2 of the container assembly 1, and includes the first washing container 210, the second washing container 220, and the third washing container 230 (i.e., is assembled by joining the first washing container 210, the second washing container 220, and the third washing container 230). The first washing container 210, the second washing container 220, and the third washing container 230 have an identical basic structure. Therefore, only the structure of the first washing container 210 is described below, and description of the structure of the second washing container 220 and the structure of the third washing container 230 is omitted.

The first washing container 210 has an approximately cylindrical shape, and extends in the longitudinal direction of the container assembly 1. The first washing container 210 includes a first insertion section 212 that is formed at one open end, the first reception section 214 that is formed at the other open end, and a cylindrical first cover section 216 that covers the first insertion section 212.

The outer diameter of the first insertion section 212 is approximately the same as the inner diameter of a second reception section 224. The inner diameter of the first reception section 214 is approximately the same as the outer diameter of the adsorption insertion section 122.

When the first insertion section 212 of the first washing container 210 is inserted and fitted into the second reception section 224 of the second washing container 220, the outer circumferential surface of the first insertion section 212 comes in close contact with (i.e., seals) the inner circumferential surface of the second reception section 224, and the first washing container 210 is joined to the second washing container 220. The first washing container 210, the second washing container 220, and the third washing container 230 are thus joined (connected) to form the washing container 200. The term “seal” used herein refers to sealing a container or the like so that at least a liquid or gas contained in the container or the like does not leak to the outside. The term “seal” used herein may include sealing a container or the like so that a liquid or gas does not enter the container or the like from the outside.

### 2-3. Elution Container

The elution container 300 has an approximately cylindrical shape, and extends in the longitudinal direction of the container assembly 1. The elution container 300 forms part of the flow channel 2 of the container assembly 1. The elution container 300 includes an elution insertion section 302 that is formed at one open end, and an elution reception section 304 that is formed at the other open end.

The inner diameter of the elution reception section 304 is approximately the same as the outer diameter of a third insertion section 232 of the third washing container 230. When the third insertion section 232 is inserted and fitted into the elution reception section 304, the outer circumferential surface of the third insertion section 232 comes in close contact with (i.e., seals) the inner circumferential surface of the elution reception section 304, and the third washing container 230 is joined to the elution container 300.

### 2-4. Reaction Container

The reaction container 400 has an approximately cylindrical shape, and extends in the longitudinal direction of the container assembly 1. The reaction container 400 forms part of the flow channel 2 of the container assembly 1. The reaction container 400 includes a reaction reception section 404 that is formed at the open end, a bottom 402 that is formed at the closed end (that is situated opposite to the open end), and a reservoir section 406 that covers the reaction reception section 404.

The inner diameter of the reaction reception section 404 is approximately the same as the outer diameter of the elution insertion section 302 of the elution container 300. When the elution insertion section 302 is inserted and fitted into the reaction reception section 404, the elution container 300 is joined to the reaction container 400.

The reservoir section 406 has a predetermined space, and is provided around the reaction reception section 404. The reservoir section 406 has a capacity sufficient to receive a liquid that overflows the reaction container 400 due to the movement of the plunger section 130.

### 3. Contents of Container Assembly, and Method for Operating Container Assembly

The contents of the container assembly 1 are described below with reference to FIG. 7A, and a method for operating the container assembly 1 is described below with reference to FIGS. 7A, 7B, 8A, and 8B. FIGS. 7A and 7B are schematic views illustrating the method for operating the container assembly 1 according to one embodiment of the invention. FIGS. 8A and 8B are schematic views illustrating the method for operating the container assembly 1 according to one embodiment of the invention. In FIGS. 7A, 7B, 8A, and 8B, each container is represented by the flow channel 2, and the external shape and the joint (junction) structure of each container are omitted so that the state of the contents can be easily understood.

### 3-1. Contents

FIG. 7A illustrates the state of the contents of the flow channel 2 when the container assembly 1 is set to the state illustrated in FIG. 1. An adsorbent 10, a first oil 20, a first washing liquid 12, a second oil 22, a second washing liquid 14, a third oil 24, a magnetic bead 30, the third oil 24, a third washing liquid 16, a fourth oil 26, an eluent 32, the fourth oil 26, and a reagent 34 are included in the flow channel 2 sequentially from the cap 110 to the reaction container 400.

The flow channel 2 has a structure in which parts (i.e., thick parts) having a large cross-sectional area (in a plane that is orthogonal to the longitudinal direction of the container assembly 1) and parts (i.e., thin parts) having a small cross-sectional area (in a plane that is orthogonal to the longitudinal direction of the container assembly 1) are provided alternately. The thin parts of the flow channel 2 respectively hold part or the entirety of the first oil 20, the second oil 22, the third oil 24, the fourth oil 26, and the eluent 32. The thin parts of the flow channel 2 have a cross-sectional area that ensures that the interface between liquids (may be fluids (hereinafter the same)) that are contiguous to each other and are immiscible with each other can be maintained within the thin part in a stable manner. Therefore, the relationship between a liquid situated within the thin part of the flow channel 2 and another liquid that is contiguous thereto can be maintained in a stable manner due to the liquid situated within the thin part. Even when the interface between a liquid situated within the thin part of the flow channel 2 and another liquid situated within the thick part of the flow channel 2 is formed within the thick part of the flow channel 2, the interface is formed at a predetermined position in a stable manner even if the interface is affected by a high impact by allowing the liquids to stand.

The thin part of the flow channel 2 is formed within the adsorption insertion section 122, the first insertion section 212, the second insertion section 222, the third insertion section 232, and the elution insertion section 302. In the elution container 300, the thin part of the flow channel 2 extends upward beyond the elution insertion section 302. Note that a liquid held within the thin part of the flow channel 2 is maintained in a stable manner even prior to assembly.

### 3-1-1. Oil

The first oil 20, the second oil 22, the third oil 24, and the fourth oil 26 include an oil, and are present in the form of a plug between the liquids contiguous thereto in the state illustrated in FIGS. 7A and 7B. A liquid that undergoes phase separation with respect to each oil (i.e., a liquid that is immiscible with each oil) is selected as the liquid contiguous to each oil so that the first oil 20, the second oil 22, the third oil 24, and the fourth oil 26 are present in the form of a plug. The first oil 20, the second oil 22, the third oil 24, and the fourth oil 26 may differ in the type of oil. An oil selected from a silicone-based oil (e.g., dimethyl silicone oil), a paraffinic oil, a mineral oil, and a mixture thereof may be used as the first oil 20, the second oil 22, the third oil 24, and the fourth oil 26, for example.

### 3-1-2. Adsorbent

The adsorbent 10 is a liquid in which the nucleic acid is adsorbed on the magnetic bead 30. For example, the adsorbent 10 is an aqueous solution that includes a chaotropic substance (material). 5 M guanidine thiocyanate, 2% Triton X-100, or 50 mM Tris-HCl (pH: 7.2) may be used as the adsorbent 10, for example. The adsorbent 10 is not particularly limited as long as the adsorbent 10 includes a chaotropic substance. A surfactant may be added to the adsorbent 10 in order to destroy a cell membrane, or denature proteins included in a cell. The surfactant is not particularly limited as long as the surfactant is normally used for extraction of a nucleic acid from a cell or the like. Specific examples of the surfactant include a nonionic surfactant such as a Triton-based surfactant (e.g., Triton-X) and a Tween-based surfactant (e.g., Tween 20), and an anionic surfactant such as sodium N-lauroyl sarcosinate (SDS). It is preferable to use a nonionic surfactant at a concentration of 0.1 to 2%. It is preferable that the adsorbent 10 include a reducing agent such as 2-mercaptoethanol or dithiothreitol. The solvent may be a buffer. It is preferable that the solvent have a pH of 6 to 8 (i.e., neutral region). It is preferable that the adsorbent 10 include a guanidine salt (3 to 7 M), a nonionic surfactant (0 to 5%), EDTA (0 to 0.2 mM), a reducing agent (0 to 0.2 M), and the like taking the above points into consideration.

The chaotropic substance is not particularly limited as long as the chaotropic substance produces chaotropic ions (i.e., monovalent anions having a large ionic radius) in an aqueous solution to increase the water solubility of hydrophobic molecules, and contributes to adsorption of the nucleic acid on the solid-phase carrier. Specific examples of the chaotropic substance include guanidine hydrochloride, sodium iodide, sodium perchlorate, and the like. It is preferable to use guanidine thiocyanate or guanidine hydrochloride that exhibits a high protein denaturation effect. These chaotropic substances are used at a different concentration. For example, guanidine thiocyanate is preferably used at a concentration of 3 to 5.5 M, and guanidine hydrochloride is preferably used at a concentration of 5 M or more.

When the chaotropic substance is present in the aqueous solution, the nucleic acid included in the aqueous solution is adsorbed on the surface of the magnetic bead 30 since it is thermodynamically advantageous for the nucleic acid to be adsorbed on a solid rather than being enclosed by water molecules.

### 3-1-3. Washing Liquid

The first washing liquid 12, the second washing liquid 14, and the third washing liquid 16 are used to wash the magnetic bead 30 on which the nucleic acid is adsorbed.

The first washing liquid 12 is a liquid that undergoes phase separation with respect to the first oil 20 and the second oil 22. It is preferable that the first washing liquid 12 be water or an aqueous solution having a low salt concentration. When using an aqueous solution having a low salt concentration as the first washing liquid 12, a buffer is preferably used as the first washing liquid 12. The salt concentration in the aqueous solution having a low salt concentration is preferably 100 mM or less, more preferably 50 mM or less, and most preferably 10 mM or less. The first washing liquid 12 may include a surfactant (see above). The pH of the first washing liquid 12 is not particularly limited. The salt that may be used for the first washing liquid 12 (buffer) is not particularly limited. It is preferable to use Tris, HEPES, PIPES, phosphoric acid, or the like. It is preferable that the first washing liquid 12 include an alcohol in such an amount that adsorption of the nucleic acid on the carrier, a reverse transcription reaction, PCR, and the like are not hindered. In this case, the alcohol concentration in the first washing liquid 12 is not particularly limited.

The first washing liquid 12 may include a chaotropic substance. For example, when the first washing liquid 12 includes guanidine hydrochloride, the magnetic bead 30 or the like can be washed while maintaining or strengthening adsorption of the nucleic acid on the magnetic bead 30 or the like.

The second washing liquid 14 is a liquid that undergoes phase separation with respect to the second oil 22 and the third oil 24. The second washing liquid 14 may have the same composition as that of the first washing liquid 12, or may have a composition differing from that of the first washing liquid 12. It is preferable that the second washing liquid 14 be a solution that substantially does not include a chaotropic substance. This is because it is preferable to prevent a situation in which a chaotropic substance is incorporated in the subsequent solution. For example, a 5 mM Tris-HCl buffer may be used as the second washing liquid 14. It is preferable that the second washing liquid 14 include an alcohol (see above).

The third washing liquid 16 is a liquid that undergoes phase separation with respect to the third oil 24 and the fourth oil 26. The third washing liquid 16 may have the same composition as that of the second washing liquid 14, or may have a composition differing from that of the second washing liquid 14. Note that the third washing liquid 16 does not include an alcohol. The third washing liquid 16 may include citric acid in order to prevent a situation in which an alcohol enters the reaction container 400.

### 3-1-4. Magnetic Bead

The magnetic bead 30 is a bead on which the nucleic acid is adsorbed. It is preferable that the magnetic bead 30 have relatively high magnetic properties so that the magnetic bead 30 can be moved using a magnet 3 that is provided outside the container assembly 1. The magnetic bead 30 may be a silica bead or a silica-coated bead, for example. The magnetic bead 30 may preferably be a silica-coated bead.

### 3-1-5. Eluent

The eluent 32 is a liquid that undergoes phase separation with respect to the fourth oil 26. The eluent 32 is present in the form of a plug that is situated between the fourth oil 26 within the flow channel 2 included in the elution container 300. The eluent 32 is a liquid with which the nucleic acid adsorbed on the magnetic bead 30 is eluted from the magnetic bead 30. The eluent 32 forms a droplet within the fourth oil 26 due to heating. For example, purified water may be used as the eluent 32. Note that the term “droplet” used herein refers to a liquid that is enclosed by a free surface.

### 3-1-6. Reagent

The reagent 34 includes a component necessary for a reaction. When effecting

PCR within the reaction container 400, the reagent 34 may include at least one of an enzyme (e.g., DNA polymerase) and a primer (nucleic acid) for amplifying the target nucleic acid (DNA) eluted into the eluent droplet 36 (see FIGS. 8A and 8B), and a fluorescent probe for detecting the amplified product. For example, the reagent 34 includes all of the primer, the enzyme, and the fluorescent probe. The reagent 34 is incompatible with the fourth oil 26. The reagent 34 is dissolved upon contact with the droplet 36 of the eluent 32 including the nucleic acid, and undergoes a reaction. The reagent 34 is present in a solid state in the lowermost part of the flow channel 2 (within the reaction container 400) in the gravitational direction. For example, a freeze-dried reagent may be used as the reagent 34.

### 3-2. Method for Operating Container Assembly

An example of the method for operating the container assembly 1 is described below with reference to FIGS. 7A, 7B, 8A, and 8B.

The method for operating the container assembly 1 includes (A) joining the adsorption container 100, the washing container 200, the elution container 300, and the reaction container 400 to assemble the container assembly 1 (hereinafter may be referred to as “step (A)”), (B) introducing a sample that includes the nucleic acid into the adsorption container 100 that holds the adsorbent 10 (hereinafter may be referred to as “step (B)”), (C) moving the magnetic bead 30 from the second washing container 220 to the adsorption container 100 (hereinafter may be referred to as “step (C)”), (D) causing the nucleic acid to be adsorbed on the magnetic bead 30 by shaking the adsorption container 100 (hereinafter may be referred to as “step (D)”), (E) moving the magnetic bead 30 on which the nucleic acid is adsorbed from the adsorption container 100 to the elution container 300 sequentially through the first oil 20, the first washing liquid 12, the second oil 22, the second washing liquid 14, the third oil 24, the third washing liquid 16, and the fourth oil 26 (hereinafter may be referred to as “step (E)”), (F) eluting the nucleic acid adsorbed on the magnetic bead 30 into the eluent 32 within the elution container 300 (hereinafter may be referred to as “step (F)”), and (G) bringing the droplet that includes the nucleic acid into contact with the reagent 34 included in the reaction container 400 (hereinafter may be referred to as “step (G)”).

Each step is described below.

Step (A) that Assembles Container Assembly 1

In the step (A), the adsorption container 100, the washing container 200, the elution container 300, and the reaction container 400 are joined to assemble the container assembly 1 so that the flow channel 2 is formed to extend from the adsorption container 100 to the reaction container 400 (see FIG. 7A). Although FIG. 7A illustrates a state in which the cap 110 is fitted to the adsorption container 100, the cap 110 is fitted to the plunger section 130 after the step (B).

More specifically, the elution insertion section 302 of the elution container 300 is inserted into the reaction reception section 404 of the reaction container 400, the third insertion section 232 of the third washing container 230 is inserted into the elution reception section 304 of the elution container 300, the second insertion section 222 of the second washing container 220 is inserted into the third reception section 234 of the third washing container 230, the first insertion section 212 of the first washing container 210 is inserted into the second reception section 224 of the second washing container 220, and the adsorption insertion section 122 of the adsorption container 100 is inserted into the first reception section 214 of the first washing container 210.

Step (B) that Introduces Sample

In the step (B), a cotton swab that holds the sample is put into the adsorbent 10 through the opening of the adsorption container 100 into which the cap 110 is fitted, and immersed in the adsorbent 10, for example. More specifically, the cotton swab is inserted into the adsorption container 100 through the opening formed at one end of the plunger section 130 that is inserted into the syringe section 120. After removing the cotton swab from the adsorption container 100, the cap 110 is fitted into the adsorption container 100 (see FIG. 7A). The sample may be introduced into the adsorption container 100 using a pipette or the like. When the sample is in the form of a paste or a solid, the sample may be put into the adsorption container 100 (or caused to adhere to the inner wall of the plunger section 130) using a spoon, tweezers, or the like. As illustrated in FIG. 7A, the syringe section 120 and the plunger section 130 are not completely filled with the adsorbent 10, and an empty space is formed on the side of the opening into which the cap 110 is fitted.

The sample includes the nucleic acid that is the target (hereinafter may be referred to as “target nucleic acid”). The target nucleic acid is either or both of deoxyribonucleic acid (DNA) and ribonucleic acid (RNA), for example. The target nucleic acid is extracted from the sample, eluted into the eluent 32 (described later), and used as a PCR template, for example. Examples of the sample include a biological sample such as blood, nasal mucus, and an oral mucous membrane, and the like.

Step (C) that Moves Magnetic Bead

In the step (C), the magnetic bead 30 that is situated between the third oil 24 and present in the form of a plug within the second washing container 220 is moved by moving the magnet 3 (that is disposed outside the container) toward the adsorption container 100 in a state in which a magnetic force is applied using the magnet 3 (see FIG. 7A).

The cap 110 and the plunger section 130 are moved in the direction away from the syringe section 120 when moving the magnetic bead 30 (or before moving the magnetic bead 30) to move the sample included in the adsorbent 10 from the plunger section 130 to the syringe section 120. The flow channel 2 that has been closed by the end section 134 communicates with the adsorbent 10 as a result of moving the plunger section 130.

The magnetic bead 30 moves upward within the flow channel 2 along with the movement of the magnet 3, and reaches the adsorbent 10 that includes the sample (see FIG. 7B).

Step (D) that Causes Nucleic Acid to be Adsorbed on Magnetic Bead

In the step (D), the adsorption container 100 is shaken. The step (D) can be efficiently performed since the opening of the adsorption container 100 is sealed with the cap 110 so that the adsorbent 10 does not leak. The target nucleic acid is thus adsorbed on the surface of the magnetic bead 30 due to the effect of the chaotropic agent. In the step (D), a nucleic acid other than the target nucleic acid and proteins may be adsorbed on the surface of the magnetic bead 30.

The adsorption container 100 may be shaken using a known vortex shaker or the like, or may be shaken manually. The adsorption container 100 may be shaken while applying a magnetic field from the outside by utilizing the magnetic properties of the magnetic bead 30.

Step (E) that Moves Magnetic Bead on which Nucleic Acid is Adsorbed

In the step (E), the magnetic bead 30 is moved through the adsorbent 10, the first oil 20, the second oil 22, the third oil 24, the fourth oil 26, the first washing liquid 12, the second washing liquid 14, and the third washing liquid 16 while applying a magnetic force generated by the magnet 3 from the outside of the adsorption container 100, the washing container 200, and the elution container 300.

For example, a permanent magnet, an electromagnet, or the like may be used as the magnet 3. The magnet 3 may be moved manually, or may be moved using a mechanical device or the like. The magnetic bead 30 is moved within the flow channel 2 through the adsorption container 100, the washing container 200, and the elution container 300 while changing the relative position of the magnet 3 by utilizing the fact that the magnetic bead 30 is attracted by a magnetic force. The speed at which the magnetic bead 30 is passed through each washing liquid is not particularly limited. The magnetic bead 30 may be moved forward and backward within an identical washing liquid along the longitudinal direction of the flow channel 2. Note that a particle or the like other than the magnetic bead 30 may be moved within the tube by utilizing gravity or a potential difference, for example.

Step (F) that Elutes Nucleic Acid

In the step (F), the nucleic acid is eluted from the magnetic bead 30 into the eluent droplet 36 within the elution container 300. In FIGS. 7A and 7B, the eluent 32 is present in the form of a plug within the thin part of the flow channel included in the elution container 300. The eluent droplet 36 moves upward within the elution container 300 (see FIGS. 8A and 8B) since the contents of the reaction container 400 expand as a result of heating the reaction container 400 while moving the magnetic bead 30. When the magnetic bead 30 has reached the eluent droplet 36 included in the elution container 300, the target nucleic acid adsorbed on the magnetic bead 30 is eluted into the eluent droplet 36 due to the effect of the eluent (see FIG. 8A).

Step (G) that Brings Droplet that Includes Nucleic Acid into Contact with Reagent 34

In the step (G), the droplet 36 that includes the nucleic acid is brought into contact with the reagent 34 that is situated in the lowermost part of the reaction container 400. Specifically, the first oil 20 is pushed downward using the end section 134 of the plunger section 130 by moving the cap 110 downward. The eluent droplet 36 into which the target nucleic acid has been eluted thus enters the reaction container 400, and comes in contact with the reagent 34 that is situated in the lowermost part of the reaction container 400 in a state in which the magnetic bead 30 to which a magnetic force generated by the magnet 3 is applied is maintained at a predetermined position (see FIG. 8B). The reagent 34 that has come in contact with the droplet 36 is dissolved, and mixed with the target nucleic acid included in the eluent. PCR that utilizes thermal cycling is thus effected, for example.

### 4. PCR Device

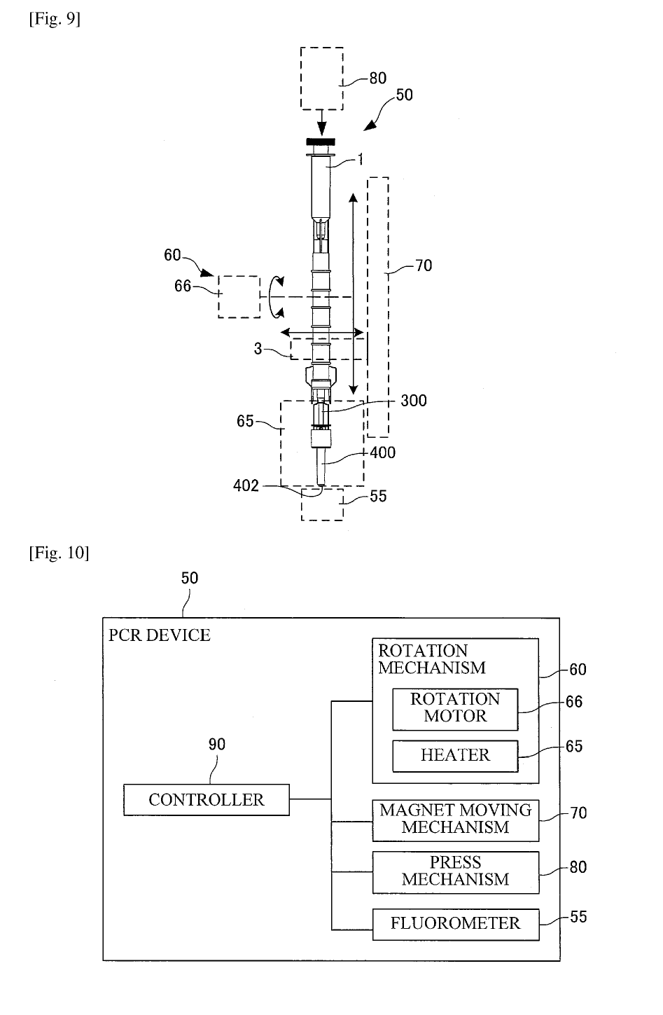

A PCR device 50 that implements a nucleic acid elution process and PCR using the container assembly 1 is described below with reference to FIGS. 9 and 10. FIG. 9 is a schematic configuration diagram illustrating the PCR device 50. FIG. 10 is a block diagram illustrating the PCR device 50.

The PCR device 50 includes a rotation mechanism 60, a magnet moving mechanism 70, a press mechanism 80, a fluorometer 55, and a controller 90.

### 4-1. Rotation Mechanism

The rotation mechanism 60 includes a rotation motor 66 and a heater 65, and rotates the container assembly 1 and the heater 65 by driving the rotation motor 66. When the container assembly 1 and the heater 65 are rotated (flipped upside down) by the rotation mechanism 60, the droplet that includes the target nucleic acid moves within the flow channel included in the reaction container 400, and subjected to thermal cycling.

The heater 65 includes a plurality of heaters (not illustrated in the drawings). For example, the heater 65 may include an elution heater, a high-temperature heater, and a low-temperature heater. The elution heater heats the eluent (that is present in the form of a plug) included in the container assembly 1 to promote elution of the target nucleic acid from the magnetic bead into the eluent. The high-temperature heater heats the upstream-side liquid within the flow channel included in the reaction container 400 to a temperature higher than that achieved by the low-temperature heater. The low-temperature heater heats the bottom 402 of the reaction container 400 (flow channel). It is possible to provide the liquid within the flow channel included in the reaction container 400 with a temperature gradient by utilizing the high-temperature heater and the low-temperature heater. The heater 65 is provided with a temperature controller, and can set the liquid within the container assembly 1 to a temperature suitable for the process according to an instruction from the controller 90.

The heater 65 has an opening that exposes the outer wall of the bottom 402 of the reaction container 400. The fluorometer 55 measures the brightness of the eluent droplet through the opening.

### 4-2. Magnet Moving Mechanism

The magnet moving mechanism 70 moves the magnet 3. The magnet moving mechanism 70 moves the magnetic bead within the container assembly 1 by moving the magnet 3 in a state in which the magnet 3 attracts the magnetic bead within the container assembly 1. The magnet moving mechanism 70 includes a pair of magnets 3, an elevating mechanism, and a swing mechanism.

The swing mechanism swings the pair of magnets 3 in the transverse direction (or the forward-backward direction) in FIG. 9. The pair of magnets 3 are disposed on either side of the container assembly 1 fitted to the PCR device 50 (see FIGS. 7A, 7B, 8A, and 8B). The distance between the magnetic bead and each magnet 3 can be reduced in the direction (transverse direction in FIG. 9) orthogonal to the flow channel of the container assembly 1. When the pair of magnets 3 are swung in the transverse direction (see the two-headed arrow), the magnetic bead within the container assembly 1 moves in the transverse direction along with the movement of the pair of magnets 3. The elevating mechanism moves the magnetic bead in the vertical direction in FIG. 9 by moving the magnet 3 in the vertical direction.

### 4-3. Press Mechanism

The press mechanism 80 presses the plunger section included in the container assembly 1. When the plunger section is pressed by the press mechanism 80, the droplet within the elution container 300 is discharged into the reaction container 400, and PCR is effected within the reaction container 400.

In FIG. 9, the press mechanism 80 is disposed above the container assembly 1 that is set to an upright state. Note that the press mechanism 80 may press the plunger section in the direction that is tilted by 45° with respect to the vertical direction, for example. This makes it possible to easily dispose the press mechanism 80 at a position at which the press mechanism 80 does not interfere with the magnet moving mechanism 70.

### 4-4. Fluorometer

The fluorometer 55 measures the brightness of the droplet within the reaction container 400. The fluorometer 55 is disposed at a position opposite to the bottom 402 of the reaction container 400. It is desirable that the fluorometer 55 be able to detect the brightness within a plurality of wavelength bands so that multiplex PCR can be implemented.

### 4-5. Controller

The controller 90 is a control section that controls the PCR device 50. The controller 90 includes a processor (e.g., CPU) and a storage device (e.g., ROM and RAM). Various programs and data are stored in the storage device. The storage device provides an area into which a program is loaded. Various processes are implemented by causing the processor to execute the program stored in the storage device.

For example, the controller 90 rotates the container assembly 1 to a predetermined rotation position by controlling the rotation motor 66. A rotation position sensor (not illustrated in the drawings) is provided to the rotation mechanism 60. The controller 90 drives and stops the rotation motor 66 corresponding to the detection results of the rotation position sensor.

The controller 90 heats the liquid within the container assembly 1 to a predetermined temperature by ON/OFF-controlling the heater 65.

The controller 90 moves the magnet 3 in the vertical direction by controlling the magnet moving mechanism 70, and swings the magnet 3 in the transverse direction in FIG. 9 corresponding to the detection results of a position sensor (not illustrated in the drawings).

The controller 90 measures the brightness of the droplet within the reaction container 400 by controlling the fluorometer 55. The measurement results are stored in a storage device (not illustrated in the drawings) included in the controller 90.

The container assembly 1 is fitted to the PCR device 50, and the steps (C) to (G) (see “3-2. Method for operating container assembly”) and PCR are effected. As described above, the biological substance extraction device may be configured so that the elution container 300 is connected to the washing container 200 (see the nucleic acid purification device 5), and the nucleic acid purification device 5 is connected to the reaction container 400 (see the container assembly 1).

### 5. Nucleic Acid Extraction Device

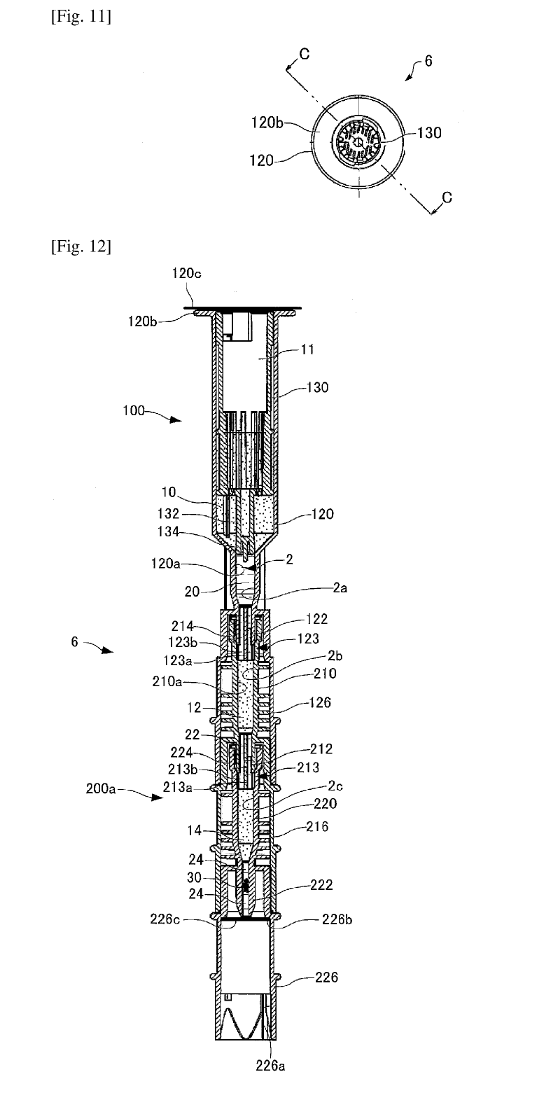

A nucleic acid extraction device 6 (i.e., biological substance extraction device) is described in detail below with reference to FIGS. 11 and 12. FIG. 11 is a plan view illustrating the nucleic acid extraction device 6 according to one embodiment of the invention. FIG. 12 is a cross-sectional view illustrating the nucleic acid extraction device 6 according to one embodiment of the invention taken along the line C-C illustrated in FIG. 11. The nucleic acid extraction device 6 is basically configured in the same manner as the adsorption container 100 and the washing container 200 included in the container assembly 1. The same elements as those of the adsorption container 100 and the washing container 200 are indicated by the same reference signs (symbols), and description of the same features as those described above in connection with the adsorption container 100 and the washing container 200 is omitted.

The nucleic acid extraction device 6 includes the adsorption container 100 (i.e., first container) that seal-tightly holds the adsorbent 10 (i.e., first liquid) and a fluid (first oil 20) that is immiscible with the adsorbent 10 within a first flow channel 2a, a washing container 200a (i.e., second container) that seal-tightly holds the first washing liquid 12 (i.e., second liquid), the second washing liquid 14 (i.e., second liquid), and a fluid (second oil 22 and third oil 24) that is immiscible with the first washing liquid 12 and the second washing liquid 14 within a second flow channel 2b and a third flow channel 2c, the adsorption container 100 and the washing container 200a being joined to form the flow channel 2 through which the target nucleic acid is moved. One end of the first flow channel 2a is inserted into one end of the second flow channel 2b so that the first flow channel 2a and the second flow channel 2b communicate with each other. In the container assembly 1 illustrated in FIGS. 1 to 8B, the washing container 200 includes three separate washing containers (i.e., first washing container 210, second washing container 220, and third washing container 230). The washing container 200a includes two separate washing containers (i.e., first washing container 210 and second washing container 220). The number of separate washing containers included in the washing container 200a may be appropriately set taking account of the application.

The adsorption container 100 includes the adsorption insertion section 122 (that is situated at one end of the first flow channel 2a), and the washing container 200 includes the first reception section 214 (that is situated at one end of the second flow channel 2b). The first flow channel 2a and the second flow channel 2b communicate with each other in a state in which the adsorption insertion section 122 is inserted into the first reception section 214.

The adsorption insertion section 122 includes an adsorption guide section 123 that includes guide members 123a and 123b that extend from the first flow channel 2a to the second flow channel 2b. Therefore, only the guide members 123a and 123b protrude from the end of the cylindrical adsorption insertion section 122 into the second flow channel 2b.

The guide members 123a and 123b form part of the flow channel 2 (through which the target nucleic acid is moved) between a first inner wall 120a of the first flow channel 2a and a second inner wall 210a of the second flow channel 2b. The guide members 123a and 123b have a plate-like shape. The front side and the back side (that are flat) of the guide members 123a and 123b are situated at a given interval from the first inner wall 120a and the second inner wall 210a, part of the flow channel 2 is formed therebetween. Each end of the guide members 123a and 123b in the widthwise direction is integrally formed with the first inner wall 120a within the adsorption insertion section 122, and comes in contact with the second inner wall 210a within the second flow channel 2b (see the vertical cross-sectional shape of the adsorption insertion section 122 and the first reception section 214 illustrated in FIG. 5).

The nucleic acid extraction device 6 is thus configured so that the magnetic bead 30 can be guided by the guide members 123a and 123b from the second flow channel 2b within the first washing container 210 toward the first flow channel 2a within the adsorption container 100 even when the adsorption insertion section 122 of the adsorption container 100 is inserted into the first reception section 214 of the first washing container 210.

A plurality of guide members 123a and 123b may be provided. In the example illustrated in FIG. 12, two guide members 123a and 123b are disposed to intersect each other. This makes it possible to improve the degree of freedom relating to phase control in the circumferential direction around the flow channel 2 of the first washing container 210 with respect to the adsorption container 100.

In the nucleic acid extraction device 6, the magnetic bead 30 is provided on the downstream side with respect to the guide members 123a and 123b. When the washing container 200a includes two separate washing containers, the magnetic bead 30 is provided in the third flow channel 2c within the second washing container 220 that is situated on the downstream side. The magnetic bead 30 provided on the downstream side with respect to the guide members 123a and 123b can be guided to the first flow channel 2a by providing the guide members 123a and 123b.

The target nucleic acid adsorption function of the magnetic bead 30 decreases when the magnetic bead 30 is stored for a long time together with a chaotropic substance. The adsorbent 10 normally includes a chaotropic substance, and it is difficult to completely prevent the movement of the chaotropic substance at a molecular level even when the adsorbent 10 is held by the first oil 20 in the shape of a plug. Therefore, it is desirable to store the magnetic bead 30 in a container that differs from the adsorption container 100 that seal-tightly holds the adsorbent 10 until the nucleic acid extraction step is performed. Since the magnetic bead 30 is a microparticle, the work efficiency decreases if the magnetic bead 30 is manually introduced into the adsorption container 100. It is possible to improve workability by utilizing the washing container 200a that holds the magnetic bead 30 in advance (i.e., nucleic acid extraction device 6). The washing liquid may include a trace amount of chaotropic substance. In such a case, it is desirable to provide that second washing container 220 in addition to the first washing container 210 that holds the first washing liquid 12 that includes the chaotropic substance, and store (provide) the magnetic bead 30 in the second washing container 220. The second washing liquid 14 held by the second washing container 220 does not include a chaotropic substance. In this case, it is necessary to provide guide members 213a and 213b to the first insertion section 212 of the first washing container 210.

Since the flow channel 2 is formed by joining the adsorption container 100, the first washing container 210, and the second washing container 220 that seal-tightly hold the liquid (as described above in connection with the container assembly 1 (and as described below)), it is possible to prevent a situation in which the magnetic bead 30 deteriorates due to a chaotropic substance.

When the magnetic bead 30 is stored in the washing container 200a that differs from the adsorption container 100, the movement of the magnetic bead 30 having a small size is hindered by the step formed at the joint between the adsorption container 100 and the washing container 200a. The adsorption guide section 123 and the first guide section 213 solves the problem in which the movement of the magnetic bead 30 is hindered by the step.

Each container used for the nucleic acid extraction device 6 is described below. Note that each container used for the nucleic acid extraction device 6 is the same as each container included in the container assembly 1 illustrated in FIGS. 1 to 8B.

### 5-1. Adsorption Container

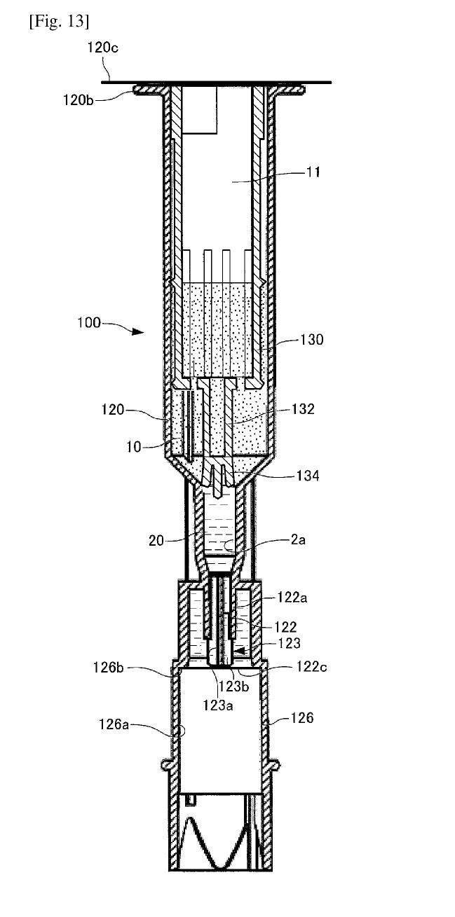

The adsorption container 100 that is used for the nucleic acid extraction device 6 is described below with reference to FIG. 13. FIG. 13 is a vertical cross-sectional view illustrating the adsorption container 100 taken along the line C-C illustrated in FIG. 11.

As illustrated in FIG. 13, the adsorption container 100 seal-tightly holds the adsorbent 10 and the first oil 20. The adsorption container 100 can be joined to the washing container 200a (first washing container 210).

The adsorption container 100 has a structure in which the plunger section 130 is inserted into the syringe section 120, and a film 120c is bonded to the upper side of a flange 120b that is situated at the upper end of the syringe section 120. The syringe section 120 includes the adsorption insertion section 122 that is situated at one end, and the flange 120b that is situated at the other end and has a circular shape that extends outward. The adsorption insertion section 122 has an approximately cylindrical shape, and has an outer wall 122a having a circular horizontal cross-sectional shape.

The syringe section 120 includes an adsorption cover section 126 that is formed around the adsorption insertion section 122, and opens downward from the upper part of the outer wall 122a. The upper end of the adsorption cover section 126 is connected to the outer wall 122a of the adsorption insertion section 122, and the lower end of the adsorption cover section 126 extends beyond the adsorption insertion section 122. An inner wall 126a of the adsorption cover section 126 has a circular step 126b at which the diameter of the inner wall 126a increases. The step 126b is situated at a position slightly lower than the lower end of the adsorption insertion section 122, and a film 122c is bonded to the surface of the step 126b.

The upper opening and the lower opening of the adsorption container 100 are respectively sealed with the film 120c and the film 122c in a state in which air 11, the adsorbent 10, and the first oil 20 are held within the flow channel 2 sequentially from the flange 120b. The adsorbent 10 and the first oil 20 are not mixed with each other. Since the flow channel 2 is sealed with the end section 134, the adsorbent 10 does not move. When the adsorption container 100 is stationary, the adsorbent 10 and the air 11 are not mixed with each other at the interface (free interface).

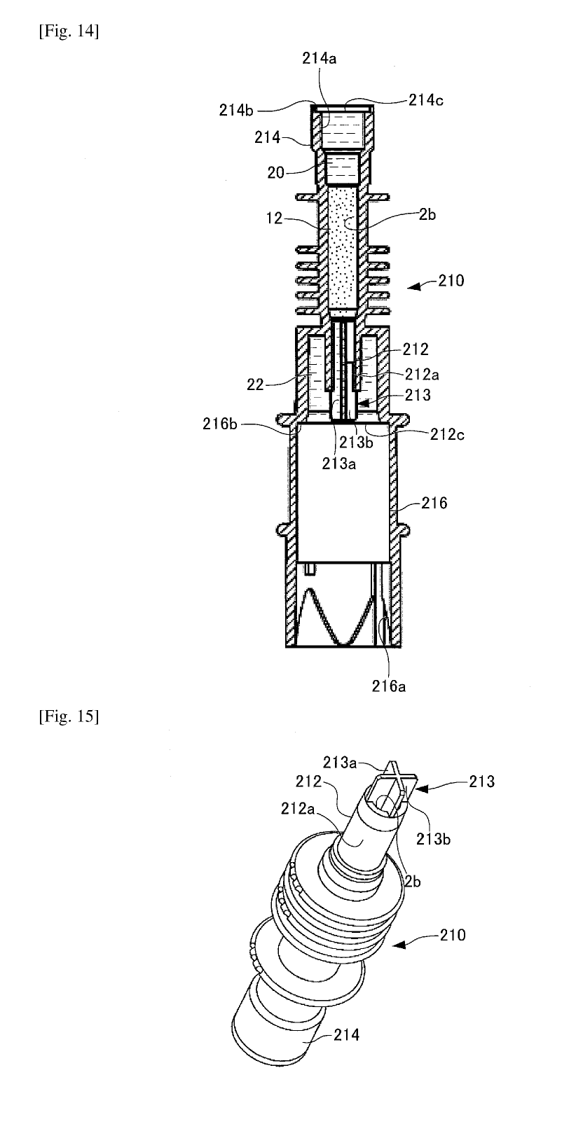

The adsorption insertion section 122 includes the adsorption guide section 123. The adsorption guide section 123 guides the movement of the magnetic bead 30. The adsorption guide section 123 includes the guide members 123a and 123b that are provided to extend from the inner wall surface of the cylindrical adsorption insertion section 122 and intersect the first flow channel 2a. The guide members 123a and 123b are integrally formed with the inner wall surface of the adsorption insertion section 122, and divides the first flow channel 2a within the adsorption insertion section 122 into a plurality of sections in the transverse direction. When the guide members 123a and 123b are two plate-like members that intersect each other, the first flow channel 2a is divided into four sections in the transverse direction. The upper end of the guide members 123a and 123b extends downward from the part in which the diameter of the first flow channel 2a decreases to be equal to the inner diameter of the adsorption insertion section 122, and the lower end of the guide members 123a and 123b protrudes from the end of the adsorption insertion section 122. The outer side of the lower end of the guide members 123a and 123b that protrudes from the end of the adsorption insertion section 122 comes in contact with the inner wall 214a (see FIG. 14) of the first reception section 214 of the first washing container 210 when the adsorption container 100 is joined to the first washing container 210. The adsorption guide section 123 is basically configured in the same manner as the first guide section 213 of the first washing container 210 (see below).

### 5-2. Washing Container

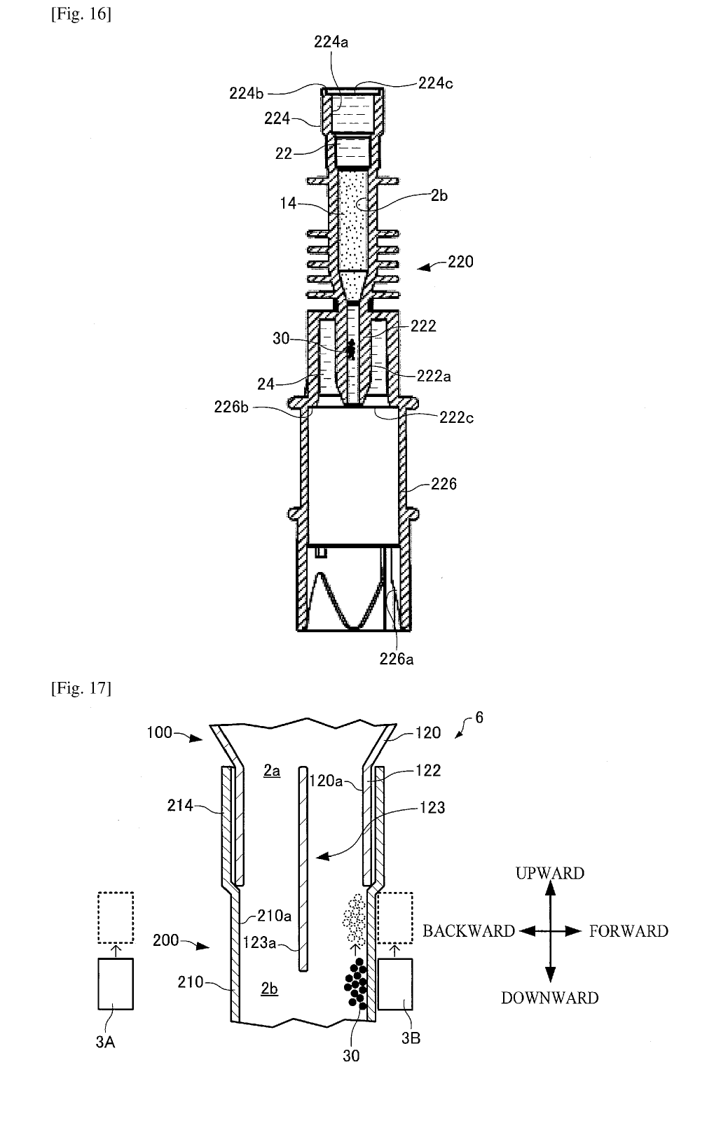

The first washing container 210 and the second washing container 220 included in the washing container 200a are described below with reference to FIGS. 14 to 16. FIG. 14 is a vertical cross-sectional view illustrating the first washing container 210 taken along the line C-C illustrated in FIG. 11. FIG. 15 is a perspective view illustrating the first washing container 210. FIG. 16 is a vertical cross-sectional view illustrating the second washing container 220 taken along the line C-C illustrated in FIG. 11.

As illustrated in FIG. 14, the first washing container 210 seal-tightly holds the first washing liquid 12 (i.e., washing liquid) and the second oil 22.

The first washing container 210 includes the first insertion section 212 (that is situated at one end of the second flow channel 2b) and the first reception section 214 (that is situated at the other end of the second flow channel 2b). The second flow channel 2b formed within the first washing container 210 extends from the first insertion section 212 to the first reception section 214. The diameter of the second flow channel 2b gradually decreases from the first reception section 214 toward the first insertion section 212.

The first insertion section 212 has an approximately cylindrical shape, and has an outer wall 212a having a circular horizontal cross-sectional shape. The first insertion section 212 includes the first guide section 213. The first guide section 213 has the same structure as that of the adsorption guide section 123. The upper end of the guide members 213a and 213b extends downward from the part in which the diameter of the second flow channel 2b decreases to be equal to the diameter of the first insertion section 212 (i.e., in the vicinity of the interface between the first washing liquid 12 and the second oil 22), and the lower end of the guide members 213a and 213b protrudes from the end of the first insertion section 212. The outer side of the lower end of the guide members 213a and 213b that protrudes from the end of the first insertion section 212 comes in contact with the inner wall 224a (see FIG. 16) of the second reception section 224 of the second washing container 220 when the first washing container 210 is joined to the second washing container 220.

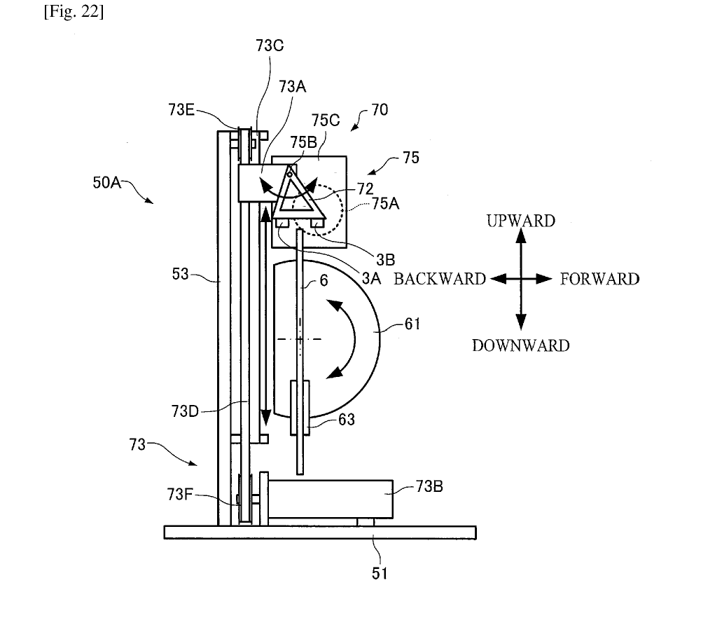

In FIG. 15, the first cover section 216 is omitted in order to clearly illustrate the shape of the first guide section 213. The guide members 213a and 213b have a plate-like shape, and are disposed to intersect each other. The guide members 213a and 213b form a crisscross horizontal cross-sectional shape. Each end of the guide members 213a and 213b in the widthwise direction is situated along the extension of the outer wall 212a of the first insertion section 212. Since the guide members 213a and 213b (i.e., a plurality of guide members) are provided, the degree of freedom relating to phase control in the circumferential direction around the second flow channel 2b and the third flow channel 2c of the second washing container 220 with respect to the first washing container 210 can be improved when joining the first washing container 210 and the second washing container 220. Specifically, when only the guide member 213a is provided, it is necessary to join the first washing container 210 and the second washing container 220 while setting the phase of the second washing container 220 with respect to the first washing container 210 so that the front side or the back side of the plate-shaped guide members 213a and 213b is necessarily situated opposite to the movement of the magnetic bead 30 in the horizontal direction (i.e., the forward-backward direction in FIG. 22). In this case, the phase of the second washing container 220 with respect to the first washing container 210 is controlled to be 180°. On the other hand, when the guide members 213a and 213b (i.e., a plurality of guide members) are provided, the phase of the second washing container 220 with respect to the first washing container 210 can be controlled to be 90°. The joining work is facilitated, and the joint structure of each container can be simplified by increasing the degree of freedom relating to phase control. This also applies to the joint between the adsorption container 100 and the first washing container 210.

As illustrated in FIG. 14, the first washing container 210 includes the first cover section 216 that is formed around the first insertion section 212, and opens downward from the upper part of the outer wall 212a. The inner wall 216a of the first cover section 216 has a circular step 216b at which the diameter of the inner wall 216a increases. The step 216b is situated at a position slightly lower than the lower end of the first insertion section 212, and a film 212c is bonded to the surface of the step 236b.

The first reception section 214 has an approximately cylindrical shape, and has an inner wall 214a having a circular horizontal cross-sectional shape. The inner wall 214a has a tubular step 214b at which the diameter of the inner wall 214a increases. The step 214b is situated in the vicinity of the upper end of first reception section 214, and a film 214c is bonded to the surface of the step 214b.

The upper opening and the lower opening of the first washing container 210 are respectively sealed with the film 214c and the film 212c in a state in which the first oil 20, the first washing liquid 12, and the second oil 22 are held within the second flow channel 2b sequentially from the first reception section 214. The first oil 20 and the second oil 22 that are seal-tightly held by the first washing container 210 hold the first washing liquid 12 in the shape of a plug.

As illustrated in FIG. 16, the second washing container 220 seal-tightly holds the second oil 22, the second washing liquid 14 (i.e., washing liquid), and the third oil 24.

The second washing container 220 includes the second insertion section 222 (that is situated at one end of the third flow channel 2c) and the second reception section 224 (that is situated at the other end of the third flow channel 2c). The third flow channel 2c formed within the second washing container 220 extends from the second insertion section 222 to the second reception section 224. The diameter of the third flow channel 2c gradually decreases from the second reception section 224 toward the second insertion section 222.

The second insertion section 222 has an approximately cylindrical shape, and has an outer wall 222a having a circular horizontal cross-sectional shape. The second insertion section 222 is not provided with a guide member.

The second washing container 220 includes a second cover section 226 that is formed around the second insertion section 222, and opens downward from the upper part of the outer wall 222a. An inner wall 226a of the second cover section 226 has a circular step 226b at which the diameter of the inner wall 226a increases. The step 226b is situated at a position slightly lower than the lower end of the second insertion section 222, and a film 222c is bonded to the surface of the step 226b.

The second reception section 224 has an approximately cylindrical shape, and has an inner wall 224a having a circular horizontal cross-sectional shape. The inner wall 224a has a tubular step 224b at which the diameter of the inner wall 224a increases. The step 224b is situated in the vicinity of the upper end of the second reception section 224, and a film 224c is bonded to the surface of the step 224b.

The upper opening and the lower opening of the second washing container 220 are respectively sealed with the film 224c and the film 222c in a state in which the second oil 22, the second washing liquid 14, the third oil 24, the magnetic bead 30, and the third oil 24 are held within the third flow channel 2c sequentially from the second reception section 224. The second oil 22 and the third oil 24 that are seal-tightly held by the second washing container 220 hold the second washing liquid 14 in the shape of a plug, and the third oil 24 holds the magnetic bead 30. Since the second washing liquid 14 does not include a chaotropic substance, it is possible to prevent a situation in which the magnetic bead 30 deteriorates due to a chaotropic substance when the second washing container 220 is sealed with the film 224c and the film 222c.

When the adsorption container 100 and the first washing container 210 are joined, or the first washing container 210 and the second washing container 220 are joined, the adsorption insertion section 122 is inserted into the first reception section 214, or the first insertion section 212 is inserted into the second reception section 224 while the insertion section 122 and the reception section 214 break the film 122c and the film 214c, or the insertion section 212 and the reception section 224 break the film 212c and the film 224c. Specifically, the first flow channel 2a included in the adsorption container 100 and the third flow channel 2c included in the second washing container 220 do not communicate with each other until the films 122c, 214c, 212c, and 224c break.

The target nucleic acid is adsorbed on the magnetic bead 30 within the adsorption container 100. Therefore, it is desirable to move the magnetic bead 30 to the adsorption container 100 promptly after the containers have been joined.

### 5-3. Operation

An operation that moves the magnetic bead 30 from the second flow channel 2b to the first flow channel 2a is described below with reference to FIGS. 17 to 20. FIGS. 17 to 20 are schematic views illustrating a method for operating the nucleic acid extraction device 6 according to one embodiment of the invention. In FIGS. 17 to 20, the adsorption cover section 126 and the guide member 123b are omitted for convenience of explanation. In FIGS. 17 to 20, the upward direction, the downward direction, the forward direction, and the backward direction are indicated by the arrows. Note that an operation that moves the magnetic bead 30 from the third flow channel 2c to the second flow channel 2b is basically the same as the operation that moves the magnetic bead 30 from the second flow channel 2b to the first flow channel 2a, and description thereof is omitted.

As illustrated in FIG. 17, each magnetic bead 30 is attracted by a magnet 3B that is situated closer to the magnetic bead 30 than a magnet 3A, and moves toward the second inner wall 210a of the second flow channel 2b that is situated in the forward direction. When the magnet 3B is moved in the upward direction, the magnetic bead 30 moves upward along the second flow channel 2b (i.e., moves to the position indicated by the broken line). If the magnetic bead 30 is continuously moved upward, the magnetic bead 30 collides with the end (step) of the adsorption insertion section 122. Specifically, the magnetic bead 30 cannot easily move beyond the step at which the flow channel narrows.

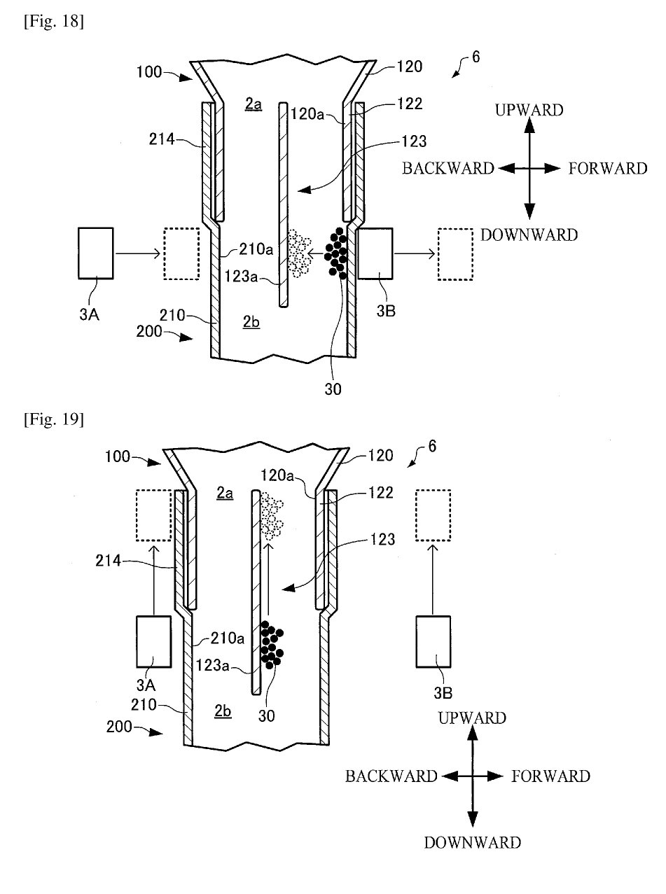

Therefore, the magnets 3A and 3B are moved in the horizontal direction (forward direction) (see FIG. 18). When the magnet 3B has moved away from the second flow channel 2b, and the magnet 3A has approached the second flow channel 2b, the magnetic bead 30 is attracted by the magnet 3A. Since the guide member 123a extends within the second flow channel 2b, the magnetic bead 30 attracted by the magnet 3A collides with the surface of the guide member 123a that is situated in the forward direction, and stops (i.e., moves to the position indicated by the broken line).

When the magnets 3A and 3B are moved upward in a state in which the magnetic bead 30 is attracted by the magnet 3A (see FIG. 19), the magnetic bead 30 moves to the first flow channel 2a along the guide member 123a (i.e., moves to the position indicated by the broken line).

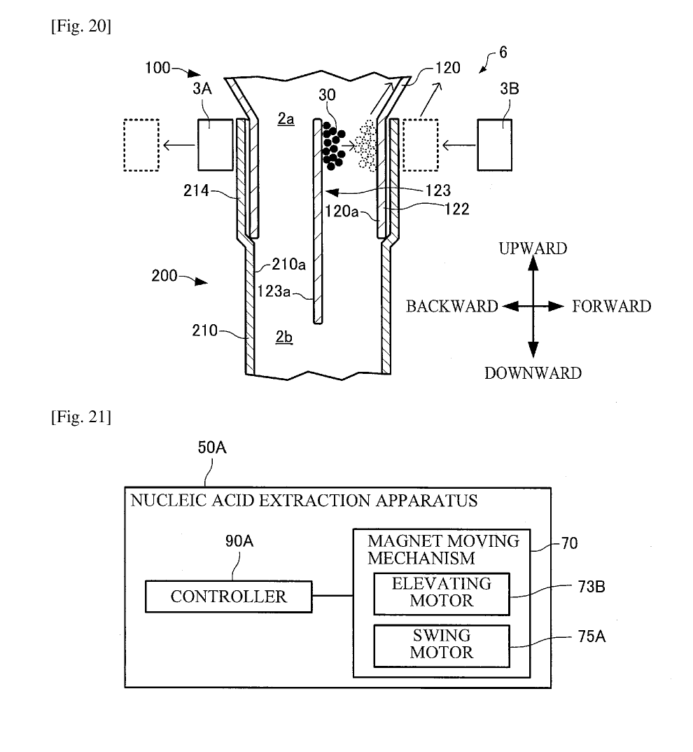

The magnets 3A and 3B are then moved in the horizontal direction (backward direction) (see FIG. 20). When the magnet 3A has moved away from the first flow channel 2a, and the magnet 3B has approached the first flow channel 2a, the magnetic bead 30 is attracted by the magnet 3B (i.e., moves to the position indicated by the broken line). When the magnet 3B is then moved upward, the magnetic bead 30 moves to the adsorption container 100 along the first flow channel 2a.

Specifically, the magnetic bead 30 can be smoothly moved by utilizing the guide member 213a while preventing a situation in which the movement of the magnetic bead 30 is hindered by a step at which the flow channel narrows.

When the target nucleic acid has been adsorbed on the magnetic bead 30 within the adsorption container 100, the magnets 3A and 3B are moved downward to move the magnetic bead 30 to the second flow channel 2b and the third flow channel 2c together with the target nucleic acid. The magnetic bead 30 can be smoothly moved downward by merely moving the magnets 3A and 3B downward since the flow channel broadens from the first flow channel 2a toward the second flow channel 2b.

### 6. Nucleic Acid Extraction Apparatus

A nucleic acid extraction apparatus 50A (i.e., biological substance extraction apparatus) is described below with reference to FIGS. 21 and 22. FIG. 21 is a block diagram illustrating the nucleic acid extraction apparatus 50A according to one embodiment of the invention. FIG. 22 is a side view illustrating the nucleic acid extraction apparatus 50A according to one embodiment of the invention. The nucleic acid extraction apparatus 50A implements a nucleic acid extraction process using the nucleic acid extraction device 6. The upward direction, the downward direction, the forward direction, and the backward direction are defined as illustrated in FIG. 22 (see the arrows). Specifically, the vertical direction when a base 51 of the nucleic acid extraction apparatus 50A is placed horizontally is referred to as “upward-downward direction”, and the upward direction and the downward direction are defined based on the gravitational direction. The direction that is perpendicular to the upward-downward direction in which the magnet 3A and 3B are moved relative to the nucleic acid extraction device 6 is referred to as “forward-backward direction”.

As illustrated in FIG. 21, the nucleic acid extraction apparatus 50A includes a magnet moving mechanism 70 that includes an elevating motor 73B a swing motor 75A, and a controller 90A.

### 6-1. Controller

The controller 90A is a control section that controls the nucleic acid extraction apparatus 50A. The controller 90A includes a processor (e.g., CPU) and a storage device (e.g., ROM and RAM). Various programs and data are stored in the storage device. The storage device provides an area into which a program is loaded. Various processes are implemented by causing the processor to execute the program stored in the storage device.

For example, the controller 90A moves the magnets 3A and 3B in the upward-downward direction by controlling the elevating motor 73B. The controller 90A swings the magnets 3A and 3B in the forward-backward direction by controlling the swing motor 75A. The elevating motor 73B and the swing motor 75A are controlled by rotating elevating motor 73B and the swing motor 75A from the initial position at a given pulse number through a pulse control process. A position sensor that detects the positions of the magnets 3A and 3B may be provided to the nucleic acid extraction apparatus 50A. In this case, the controller 90A drives or stops the elevating motor 73B and the swing motor 75A corresponding to the detection results of the position sensor.

As illustrated in FIG. 22, the nucleic acid extraction apparatus 50A includes a holding section 63 that holds the nucleic acid extraction device 6, and the magnet moving mechanism 70 that moves the magnets 3A and 3B along the nucleic acid extraction device 6 that is held by the holding section 63.

The holding section 63 is positioned at the lower end of a rotating body 61, and holds the nucleic acid extraction device 6 at a given position with respect to the rotating body 61. The rotating body 61 can be rotated in the direction indicated by the double-headed arrow. For example, the nucleic acid extraction device 6 is inserted into (held by) the holding section 63 in a state in which the rotating body 61 is rotated clockwise by −30°. After rotating the rotating body 61 counterclockwise by +30° to set the nucleic acid extraction device 6 to the initial state illustrated in FIG. 22, the magnet moving mechanism 70 is operated.

### 6-2. Magnet Moving Mechanism

The magnet moving mechanism 70 moves the magnets 3A and 3B. The magnet moving mechanism 70 allows the magnetic bead 30 within the nucleic acid extraction device 6 to be attracted by the magnets 3A and 3B, and moves the magnetic bead 30 within the nucleic acid extraction device 6 by moving the magnets 3A and 3B. The magnet moving mechanism 70 includes the magnets 3A and 3B that make a pair, an elevating mechanism 73, and a swing mechanism 75.

The magnets 3A and 3B are members that attract the magnetic bead 30. A permanent magnet, an electromagnet, or the like may be used as magnets 3A and 3B. The magnets 3A and 3B are held by an arm 72 so that the magnets 3A and 3B are situated at an almost identical position in the upward-downward direction and are situated opposite to each other in the forward-backward direction through the nucleic acid extraction device 6.

The elevating mechanism 73 moves the magnets 3A and 3B in the upward-downward direction. The elevating mechanism 73 includes a carriage 73A that moves in the upward-downward direction, and the elevating motor 73B. The carriage 73A is a member that can move in the upward-downward direction. The carriage 73A is guided by a carriage guide 73C in the upward-downward direction, the carriage guide 73C being provided to a side wall 53 that vertically extends from the base 51. The elevating motor 73B is a motor that moves the carriage 73A in the upward-downward direction. The elevating motor 73B moves the carriage 73A to a given position in the upward-downward direction according to instructions output from the controller 90. The elevating motor 73B moves the carriage 73A in the upward-downward direction using pulleys 73E and 73F provided to the upper end and the lower end of the side wall 53, and a belt 73D that is provided around the pulleys 73E and 73F.

The swing mechanism 75 swings the magnets 3A and 3B in the forward-backward direction. When the magnets 3A and 3B are swung in the forward-backward direction, the interval between each magnet and the nucleic acid extraction device 6 changes alternately. Since the magnetic bead 30 is attracted by one of the magnets 3A and 3B that is situated closer to the magnetic bead 30, the magnetic bead 30 within the nucleic acid extraction device 6 is moves in the forward-backward direction by swinging the magnets 3A and 3B in the forward-backward direction.

The swing mechanism 75 includes the swing motor 75A and a holding plate 75C.

The holding plate 75C is secured on the carriage 73A, and can be moved in the upward-downward direction together with the carriage 73A. The holding plate 75C holds the swing motor 75A. When power generated by the swing motor 75A is transmitted to a swing rotation shaft 75B through a gear (not illustrated in FIG. 22), the arm 72 that holds the magnets 3A and 3B is rotated around the swing rotation shaft 75B relative to the carriage 73A. The swing mechanism 75 swings the magnets 3A and 3B in the forward-backward direction so that the magnets 3A and 3B do not come in contact with the nucleic acid extraction device 6. Note that a horizontal moving mechanism or the like may be provided instead of the swing mechanism 75 as long as the magnets 3A and 3B can be moved in the forward-backward direction.

### 6-3. Nucleic Acid Extraction Process

The nucleic acid extraction process includes (a) joining the adsorption container 100, the first washing container 210, and the second washing container 220 to assemble the nucleic acid extraction device 6, (b) securing the nucleic acid extraction device 6 on the holding section 63 of the nucleic acid extraction apparatus 50A (c) introducing a sample that includes a nucleic acid into the adsorption container 100 that holds the adsorbent 10, (d) rotating the rotating body 61 to set the nucleic acid extraction device 6 to the initial position, (e) moving the magnetic bead 30 from the second washing container 220 to the adsorption container 100, (f) causing the nucleic acid to be adsorbed on the magnetic bead 30 by shaking the adsorption container 100, and (g) moving the magnetic bead 30 on which the nucleic acid is adsorbed from the adsorption container 100 sequentially through the first oil 20, the first washing liquid 12, the second oil 22, the second washing liquid 14, and the third oil 24.

In the step (e), the magnetic bead 30 within the second washing container 220 is moved to the adsorption container 100 along the guide members 123a, 123b, 213a, and 213b by moving the magnets 3A and 3B using the magnet moving mechanism 70 (as described above with reference to FIGS. 17 to 20). The magnetic bead 30 can be moved from the third flow channel 2c to the first flow channel 2a by thus moving the magnetic bead 30 along the guide members 123a, 123b, 213a, and 213b by moving the magnets 3A and 3B using the magnet moving mechanism 70.

When the nucleic acid extraction device 6 includes the third washing container 230 and the elution container 300 (as described above with reference to FIGS. 1 to 8B), the nucleic acid extraction process may include (g′) moving the magnetic bead 30 on which the nucleic acid is adsorbed to the elution container 300 through the third washing liquid 16 and the fourth oil 26, and (h) eluting the nucleic acid from the magnetic bead 30 into the eluent 32 within the elution container 300.

The target nucleic acid can be eluted from the magnetic bead 30 by thus causing the target nucleic acid to be adsorbed on the magnetic bead 30 that has moved to the adsorption container 100, and moving the magnetic bead 30 along the flow channel 2 within the washing container 200a (or the washing container 200) and the elution container 300 using the magnet moving mechanism 70.

The invention is not limited to the above embodiments. Various modifications and variations may be made without departing from the scope of the invention. For example, the invention includes various other configurations that are substantially the same as the configurations described in connection with the above embodiments (e.g., a configuration having the same function, method, and results, or a configuration having the same objective and results). Although the above embodiments have been described taking an example in which the first container is the adsorption container, and the second container is the washing container, another container may be used as the first container and the second container. For example, when the first container is the washing container 200, and the second container is the elution container 300, the magnetic bead 30 from which the target nucleic acid has been eluted can be moved upward from the elution container 300 to the washing container 200 by providing the guide members 123a and 123b to the joint between the washing container 200 and the elution container 300. The invention also includes a configuration in which an unsubstantial element described in connection with the above embodiments is replaced by another element. The invention also includes a configuration having the same effects as those of the configurations described in connection with the above embodiments, or a configuration capable of achieving the same objective as that of the configurations described in connection with the above embodiments. The invention further includes a configuration in which a known technique is added to the configurations described in connection with the above embodiments.

1. A biological substance extraction device comprising:
a flow channel through which a biological substance is moved, the flow channel being formed by joining a first container that includes a first flow channel and seal-tightly holds a first liquid and a fluid that is immiscible with the first liquid within the first flow channel, and a second container that includes a second flow channel and seal-tightly holds a second liquid and a fluid that is immiscible with the second liquid within the second flow channel,
one end of the first flow channel being inserted into one end of the second flow channel so that the first flow channel and the second flow channel communicate with each other,
the first container including a guide member that extends from the first flow channel to the second flow channel when the first flow channel and the second flow channel communicate with each other, and
the guide member forming part of the flow channel between a first inner wall of the first flow channel and a second inner wall of the second flow channel.

2. The biological substance extraction device as defined in claim 1,
the guide member having a plate-like shape, and
a plurality of the guide members being provided to intersect each other.

3. The biological substance extraction device as defined in claim 1,
a substance-binding solid-phase carrier being provided on a downstream side of the guide member within the flow channel through which the biological substance is moved.

4. The biological substance extraction device as defined in claim 1,
the first container being an adsorption container,
the second container being a washing container,
the first liquid being an adsorbent, and
the second liquid being a washing liquid.

5. A biological substance extraction apparatus comprising:
a holding section that holds the biological substance extraction device as defined in claim 4; and
a magnet moving mechanism that moves a magnet along the biological substance extraction device that is held by the holding section,
the magnet moving mechanism moving a substance-binding solid-phase carrier provided within the washing container to the adsorption container along the guide member by moving the magnet.

6. The biological substance extraction apparatus as defined in claim 5,
the biological substance extraction device further comprising an elution container that is connected to the other end of the second flow channel,
the elution container holding an eluent that is a liquid with which the biological substance is eluted from the substance-binding solid-phase carrier, and
the magnet moving mechanism moving the substance-binding solid-phase carrier through the adsorption container, the washing container, and the elution container along the flow channel by moving the magnet to elute the biological substance from the substance-binding solid-phase carrier.
